(19) United States
(12) Patent Application Publication
(10) Pub. No.: US 2023/0051158 A1
(43) Pub. Date: Feb. 16, 2023

(54) Disposable Sport Jock Device

(71) Applicants: Adaliah Holley, Desoto, TX (US); Andriah Holley-Smith, Houston, TX (US)

(72) Inventors: Adaliah Holley, Desoto, TX (US); Andriah Holley-Smith, Houston, TX (US)

(21) Appl. No.: 17/884,850

(22) Filed: Aug. 10, 2022

Related U.S. Application Data
(60) Provisional application No. 63/232,226, filed on Aug. 12, 2021.

(51) Int. Cl. A41B 9/00; A41D 1/06; A41D 31/12

(52) U.S. Cl. CPC A41B 9/001; A41D 1/06; A41D 31/125

(57) ABSTRACT

The present invention relates to a novel disposable undergarment device. The device is designed to ensure accidental body leaks are absorbed prior to staining clothing and causing an embarrassing situation. The device comprises absorbent, disposable boxer briefs, standard briefs, and/or female undergarments that can be worn during athletic events and other activities. The sides of the undergarments are expandable and attached via two-sided tape, as well as an elastic waistband. The inside of the undergarments comprises an absorbent padding which is constructed using a fluffy, paper-like material, such as cellulose fiber, which is designed to enhance the fit of the padding and help stop leaks.

### CROSS-REFERENCE TO RELATED APPLICATION

The present application claims priority to, and the benefit of, U.S. Provisional Application No. 63/232,226, which was filed on Aug. 12, 2021, and is incorporated herein by reference in its entirety.

### FIELD OF THE INVENTION

The present invention relates generally to the field of disposable undergarment devices for men and women. More specifically, the present invention relates to a disposable undergarment device that provides users with a disposable undergarment capable of being worn underneath clothing during athletic events. Accordingly, the present disclosure makes specific reference thereto. Nonetheless, it is to be appreciated that aspects of the present invention are also equally applicable to other like applications, devices, and methods of manufacture.

### BACKGROUND

By way of background, this invention relates to improvements in disposable undergarments of the type which are used during athletic events to prevent worry of embarrassing accidents during athletic events and other activities. For example, athletes, especially younger children, may accidentally urinate during practices and/or games. Further, embarrassing wet spots can develop and leave children and adults extremely uncomfortable. Additionally, women may have trouble containing leaks during their period and while participating in athletic activities. Furthermore, while disposable underwear is widely accepted for ease of use, such underwear is often cumbersome, uncomfortable, audible, and aesthetically unattractive.

Thus, it is necessary for users and athletes to have an improved, disposable undergarment capable of being worn during athletic events and other activities. Generally, conventional undergarments do not prevent embarrassing leaks and do not prevent clothing stains. Therefore, users require a disposable undergarment which offers a way to reduce the chance of bodily fluid leaks.

Therefore, there exists a long-felt need in the art for a disposable undergarment device that provides users with an improved, disposable undergarment capable of being worn underneath clothing during athletic events. There is also a long-felt need in the art for a disposable undergarment device that ensures accidental urination can be absorbed prior to staining clothing and causing an embarrassing situation. Further, there is a long-felt need in the art for a disposable undergarment device that includes expandable sides attached to two-sided adhesive tape and an elastic waistband that can be firmly secured underneath clothing. Moreover, there is a long-felt need in the art for a device that prevents young children from having an accident and being forced to abandon their sporting event to eliminate the worry of embarrassment. Further, there is a long-felt need in the art for a disposable undergarment device that offers a way to reduce the chance of bodily fluid leaks until visiting a bathroom or changing clothes can occur. Finally, there is a long-felt need in the art for a disposable undergarment device that provides a disposable undergarment for athletes and other users.

The subject matter disclosed and claimed herein, in one embodiment thereof, comprises a disposable undergarment device. The device is designed to ensure accidental body leaks are absorbed prior to staining clothing and causing an embarrassing situation. The device comprises absorbent, disposable boxer briefs, standard briefs, and/or female undergarments that can be worn during athletic events and other activities. The sides of the undergarments are expandable and attached via two-sided tape, as well as an elastic waistband. The inside undergarments comprise an absorbent padding which is constructed using a fluffy, paper-like material, such as cellulose fiber, which is designed to enhance the fit of the padding and help stop leaks. Padding in the undergarment is focused on the front for males and focused between the thigh area for females.

In this manner, the disposable undergarment device of the present invention accomplishes all of the foregoing objectives and provides users with a device that ensures accidental urination can be absorbed prior to staining clothing and causing an embarrassing situation. The device offers a way to reduce the chance of bodily fluid leaks until a change of clothing can occur. The device prevents users from having an accident and being forced to abandon their sporting event or other activity.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a disposable undergarment device. The device is designed to ensure accidental body leaks are absorbed prior to staining clothing and causing an embarrassing situation. The device comprises absorbent, disposable boxer briefs, standard briefs, and/or female undergarments that can be worn during athletic events and other activities. The sides of the undergarments are expandable and attached via two-sided tape, as well as an elastic waistband. The inside undergarments comprise an absorbent padding which is constructed using a fluffy, paper-like material, such as cellulose fiber, which is designed to enhance the fit of the padding and help stop leaks. Padding in the undergarment is focused on the front for males and focused between the thigh area for females.

Typically, disposable undergarments are of standard design, wherein the underwear is specified by gender and end use application. For example, the briefs and/or female undergarments of the present invention are set to various sizes, i.e., small, medium, large, etc., in order to adapt to various body types. Further, the briefs and other underwear styles can be manufactured in children's sizes as well. In general, the disposable undergarment is preferably composed of one or more pieces that are thermally bonded at the seams, mechanically sewn, or bonded with an adhesive. Each disposable undergarment comprises a waist opening at the top of the undergarment and a pair of leg openings at the bottom of the undergarment. The disposable undergarment can optionally contain stretchable portions around the waist and leg openings.

In one embodiment, the disposable undergarment comprises a front panel and a back panel with an interconnecting crotch region, wherein the front panel, back panel, and interconnecting crotch region are mechanically secured together at their lateral ends (i.e., the front and back panels are joined at opposite seams and the crotch area of each panel is joined at the bottom seam). Alternatively, the disposable undergarment may be structurally unitary, generally have an hourglass shape, and the front and back panels may be integrally joined with the interconnecting crotch region. The disposable undergarment is then formed by joining the front and back panels at opposite seams.

In one embodiment, the disposable undergarment comprises two elastic leg openings and an elastic waistband. The elastic waistband includes expandable sides to allow a customizable fit for users. Once in the proper position for a user, the expandable sides are secured via two-sided tape, or any other suitable securing means as is known in the art. Further, the disposable undergarment has a layered structure, wherein a soft hydrophilic rayon fabric layer is disposed inside the underwear facing the skin but is hydrophobic. The disposable undergarment of this embodiment also contains a separate cellulosic defense panel for moisture treatment. Typically, the separate cellulosic defense panel for moisture treatment can be positioned toward the front for males and between the thighs for females. Any suitable layers of cellulosic defense panels can be utilized as is known in the art and depending on the needs and/or wants of a user. Users can add more padding, i.e., cellulosic defense panels, depending on their level of activity or length of time participating, etc.

It is to be understood that the present invention is by no means limited to the particular construction herein disclosed and/or shown in the drawings, but also comprises any modifications or equivalents within the scope of the disclosure. Further, the disposable undergarment device can be used for anyone, not just athletes. Anyone needing protection from bodily leaks can utilize the device as needed, such as people who are traveling, or a user recovering from prosrate cancer, etc.

In yet another embodiment, the nonwoven fabric of the present invention is formed from natural fibers, synthetic fibers, or a combination of natural and synthetic fibers. The fibers can have a finite staple length and can be continuous filaments and blends thereof. The synthetic fibers are selected from thermosetting polymers such as polyacrylates, or thermoplastic polymers, including polyamides, polyesters, or polyolefins such as polypropylene or polyethylene, derivatives thereof, and combinations thereof. The synthetic fibers of the present invention can include any fiber having a multi-component configuration, such as parallel or shell-core, as well as geometric variants. The natural fibers of the present invention are cellulosic in nature, such as cotton, wood pulp, or rayon.

Nonwoven fabrics used in accordance with the present invention must be durable and able to withstand sustained physical exercise loads associated with daily activities. The fabric must also have the appropriate drape and feel so that it conforms to the body curve. In addition, the fabric must have hydrophilic and hydrophobic sites to absorb moisture and repel water as needed. The amount and position of the hydrophilic and hydrophobic sites can vary depending on the needs and/or wants of a user or based on the activity the user is participating in, as well as whether the user is male, female, and/or a child.

The nonwoven fabrics of the present invention can be composite, laminated, monolayer or multilayer, to introduce auxiliary parts suitable for limited urinary contamination and/or absorption mechanisms, into the undergarment. Nonwovens can also be imaged with protrusions or swirls, etc., and can also form openings, or utilize subsequent dyeing and printing, or utilize colored fibers during the manufacturing stage, etc.

Further, disposable nonwoven underwear has skin health and hygiene benefits. The breathability and moisture absorption properties of nonwovens used in disposable underwear provide a comfortable environment for the skin. Nonwovens help keep the skin oxygenated and dry, which is advantageous for people with an active lifestyle.

In yet another embodiment, the disposable undergarment device comprises a plurality of indicia.

In yet another embodiment, a method of providing users with a disposable undergarment capable of being worn underneath clothing during athletic events is described. The method includes the steps of positioning the disposable undergarment around a user's legs and waist. The method also comprises securing the expandable sides via two-sided tape and an elastic waistband. Further, the method comprises wearing the disposable undergarment during an athletic event or other activity. Finally, disposing of the undergarment after use.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front perspective view of one potential embodiment of the disposable undergarment device of the present invention in accordance with the disclosed architecture;

FIG. 2 illustrates a side perspective view of one potential embodiment of the disposable undergarment device of the present invention in accordance with the disclosed architecture;

FIG. 3 illustrates a back perspective view of one potential embodiment of the disposable undergarment device of the present invention in accordance with the disclosed architecture;

FIG. 4 illustrates a side perspective view of one potential embodiment of the disposable undergarment device of the present invention in accordance with the disclosed architecture; and

FIG. 5 illustrates a flowchart showing one potential method of providing a user with a disposable undergarment capable of being worn underneath clothing during athletic events in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a disposable undergarment device that provides users with an improved, disposable undergarment capable of being worn underneath clothing during athletic events. There is also a long-felt need in the art for a disposable undergarment device that ensures accidental urination can be absorbed prior to staining clothing and causing an embarrassing situation. Further, there is a long-felt need in the art for a disposable undergarment device that includes expandable sides attached to two-sided adhesive tape and an elastic waistband that can be firmly secured underneath clothing. Moreover, there is a long-felt need in the art for a device that prevents young children from having an accident and being forced to abandon their sporting event to eliminate the worry of embarrassment. Further, there is a long-felt need in the art for a disposable undergarment device that offers a way to reduce the chance of bodily fluid leaks until visiting a bathroom or changing clothes can occur. Finally, there is a long-felt need in the art for a disposable undergarment device that provides a disposable undergarment for athletes and other users.

The present invention, in one exemplary embodiment, is a novel disposable undergarment device. The device is designed to ensure accidental body leaks are absorbed prior to staining clothing and causing an embarrassing situation. The device comprises absorbent, disposable boxer briefs, standard briefs, and/or female undergarments that can be worn during athletic events and other activities. The sides of the undergarments are expandable and attached via two-sided tape, as well as an elastic waistband. The inside undergarments comprise an absorbent padding which is constructed using a fluffy, paper-like material, such as cellulose fiber, which is designed to enhance the fit of the padding and help stop leaks. The present invention also includes a novel method of providing users with a disposable undergarment capable of being worn underneath clothing during athletic events. The method includes the steps of positioning the disposable undergarment around a user's legs and waist. The method also comprises securing the expandable sides via two-sided tape and an elastic waistband. Further, the method comprises wearing the disposable undergarment during an athletic event or other activity. Finally, disposing of the undergarment after use.

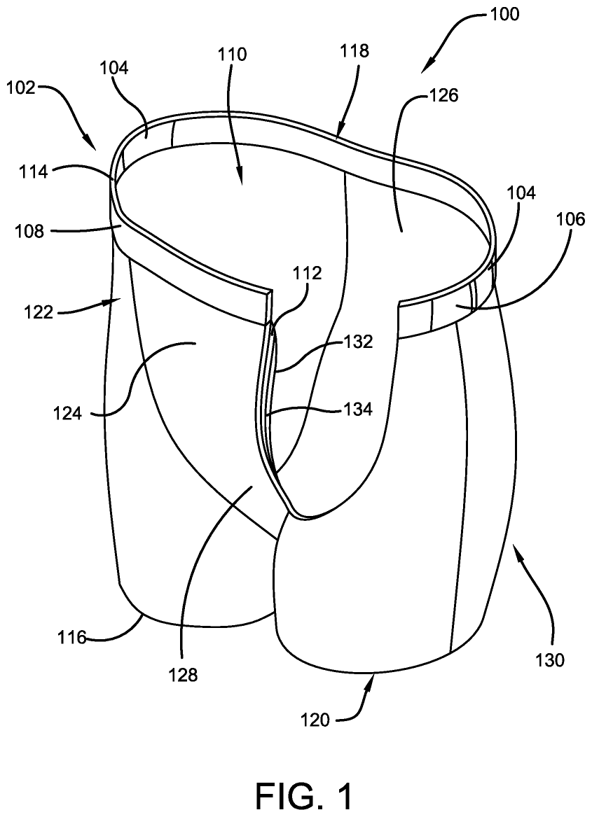

Referring initially to the drawings, FIG. 1 illustrates a perspective view of one embodiment of the disposable undergarment device 100 of the present invention. In the present embodiment, the disposable undergarment device 100 is an improved disposable undergarment device that is capable of being worn underneath clothing during athletic events to prevent bodily leaks. The device 100 is especially designed to allow any suitable user as is known in the art, to be protected from bodily leaks. More specifically, the device 100 comprises absorbent, disposable boxer briefs, standard briefs, and/or female undergarments 102 that can be worn during athletic events and other activities. The sides 104 of the undergarments 102 are expandable and attached via two-sided tape 106, as well as an elastic waistband 108. The inside 110 of the undergarments 102 comprise an absorbent padding 112 which is constructed using a fluffy, paper-like material, such as cellulose fiber, which is designed to enhance the fit of the padding 112 and help stop leaks.

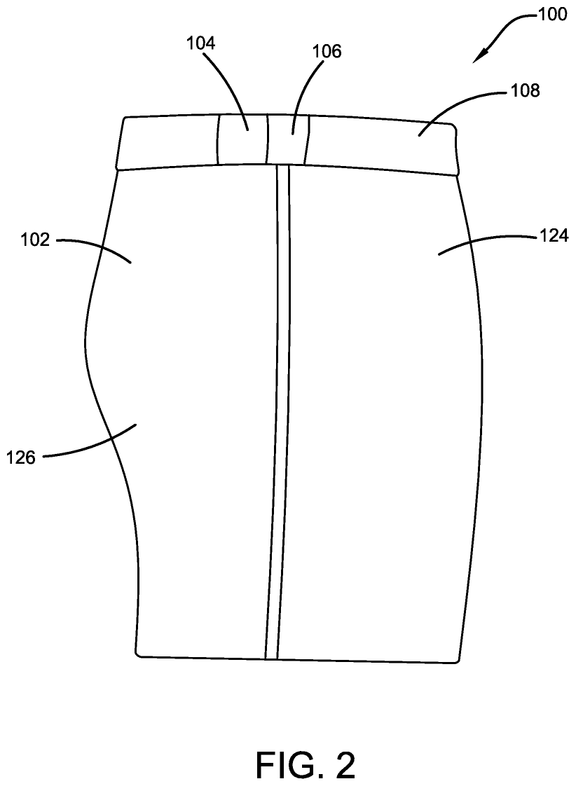

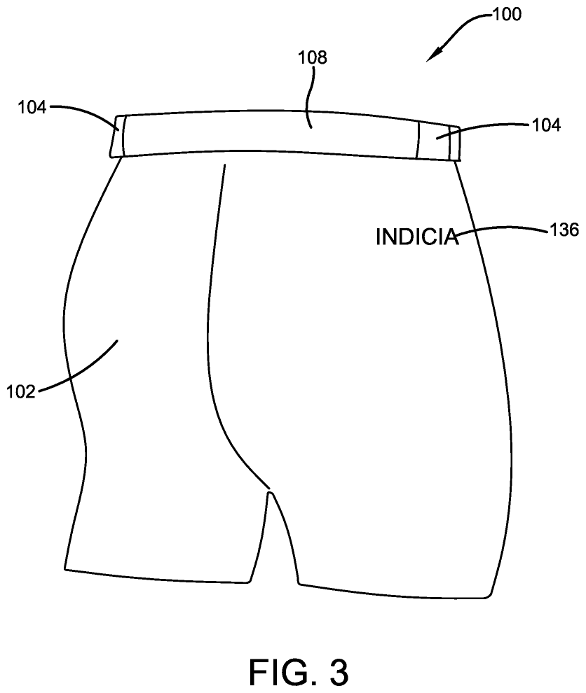

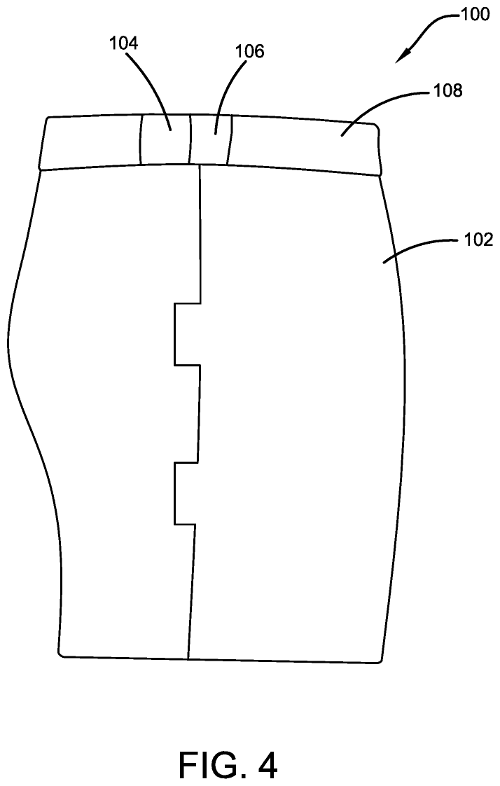

As shown in FIGS. 1-4, typically, disposable undergarments 102 are of standard design, wherein the underwear 102 is specified by gender and end use application. For example, the briefs and/or female undergarments 102 of the present invention are set to various sizes, i.e., small, medium, large, etc., in order to adapt to various body types. Further, the briefs and other underwear styles 102 can be manufactured in children's sizes as well. Additionally, any suitable shape and size of undergarment 102 can be utilized as is known in the art and based on the needs and/or wants of a user. In general, the disposable undergarment 102 is preferably composed of one or more pieces that are thermally bonded at the seams, mechanically sewn, or bonded with an adhesive. Each disposable undergarment 102 comprises a body portion 122, a waist opening 114 at the top 118 of the undergarment 102, and a pair of leg openings 116 at the bottom 120 of the undergarment 102. The disposable undergarment 102 can optionally contain stretchable portions around the waist 114 and leg openings 116.

In one embodiment, the disposable undergarment 102 comprises a front panel 124 and a back panel 126 with an interconnecting crotch region 128, wherein the front panel 124, back panel 126, and interconnecting crotch region 128 are mechanically secured together at their lateral ends (i.e., the front 124 and back 126 panels are joined at opposite seams and the crotch area 128 of each panel is joined at the bottom seam). Alternatively, the disposable undergarment 102 may be structurally unitary 130 and generally have an hourglass shape, and the front 124 and back 126 panels may be integrally joined with the interconnecting crotch region 128. The disposable undergarment 102 is then formed by joining the front 124 and back 126 panels at opposite seams.

In another embodiment, the disposable undergarment 102 comprises two elastic leg openings 116 and an elastic waistband 108. The elastic waistband 108 can be any suitable elastic waistband as is known in the art and can be formed via standard methods of inserting elastic into the waistband area. The disposable undergarment 102 can be manufactured with only an elastic waistband 108, allowing the undergarment 102 to be stretchy and comfortable for users. The elastic waistband 108 can be utilized to allow the user to pull on and off the undergarment 102 with ease, as well as allow the user to have a snug, customized fit when wearing the undergarment 102. The leg openings 116 do not have to be elasticized and the undergarment 102 can be manufactured with only the waistband 108 being elasticized, depending on the needs and/or wants of a user.

In a further embodiment, the elastic waistband 108 includes expandable sides 104 to allow a customizable fit for users. Once in the proper position for a user, the expandable sides 104 are secured via two-sided tape 106, or any other suitable securing means as is known in the art. Further, the disposable undergarment 102 has a layered structure, wherein a soft hydrophilic rayon fabric layer 132 is disposed inside the undergarment 102 facing the skin but is hydrophobic. The disposable undergarment 102 of this embodiment also contains a separate cellulosic defense panel 134 for moisture treatment. Typically, the separate cellulosic defense panel 134 for moisture treatment can be positioned toward the front for males and between the thighs for females. Any suitable layers of cellulosic defense panels 134 can be utilized as is known in the art and depending on the needs and/or wants of a user. Users can add more padding 112, i.e., cellulosic defense panels 134, depending on their level of activity or length of time participating, etc.

It is to be understood that the present invention is by no means limited to the particular construction herein disclosed and/or shown in the drawings, but also comprises any modifications or equivalents within the scope of the disclosure. Further, the disposable undergarment device 100 can be used for anyone, not just athletes. Anyone needing protection from bodily leaks can utilize the device 100 as needed, such as people who are traveling, or a user recovering from prostate cancer, etc.

In yet another embodiment, the nonwoven fabric of the present invention is formed from natural fibers, synthetic fibers, or a combination of natural and synthetic fibers. The fibers can have a finite staple length and can be continuous filaments and blends thereof. The synthetic fibers are selected from thermosetting polymers such as polyacrylates, or thermoplastic polymers including polyamides, polyesters, or polyolefins such as polypropylene or polyethylene, derivatives thereof, and combinations thereof. The synthetic fibers of the present invention can include any fiber having a multi-component configuration, such as parallel or shell-core, as well as geometric variants. The natural fibers of the present invention are cellulosic in nature, such as cotton, wood pulp, or rayon.

Nonwoven fabrics used in accordance with the present invention must be durable and able to withstand sustained physical exercise loads associated with daily activities. The fabric must also have the appropriate drape and feel so that it conforms to the body curve. In addition, the fabric must have hydrophilic and hydrophobic sites to absorb moisture and repel water as needed. The amount and position of the hydrophilic and hydrophobic sites can vary depending on the needs and/or wants of a user or based on the activity the user is participating in, as well as whether the user is male, female, and/or a child.

The nonwoven fabrics of the present invention can be composite, laminated, monolayer or multilayer, to introduce auxiliary parts suitable for limited urinary contamination and/or absorption mechanisms, into the undergarment 102. Nonwovens can also be imaged with protrusions or swirls, etc., and can also form openings, or utilize subsequent dyeing and printing, or utilize colored fibers during the manufacturing stage, etc.

Further, disposable nonwoven underwear has skin health and hygiene benefits. The breathability and moisture absorption properties of nonwovens used in disposable underwear provide a comfortable environment for the skin. Nonwovens help keep the skin oxygenated and dry, which is advantageous for people with an active lifestyle.

In yet another embodiment, the disposable undergarment device 100 comprises a plurality of indicia 136. The undergarment 102 of the device 100 may include advertising, a trademark, or other letters, designs, or characters, printed, painted, stamped, or integrated into the undergarment 102, or any other indicia 136 as is known in the art. Specifically, any suitable indicia 136 as is known in the art can be included, such as, but not limited to, patterns, logos, emblems, images, symbols, designs, letters, words, characters, animals, advertisements, brands, etc., that may or may not be undergarment or leak brand related.

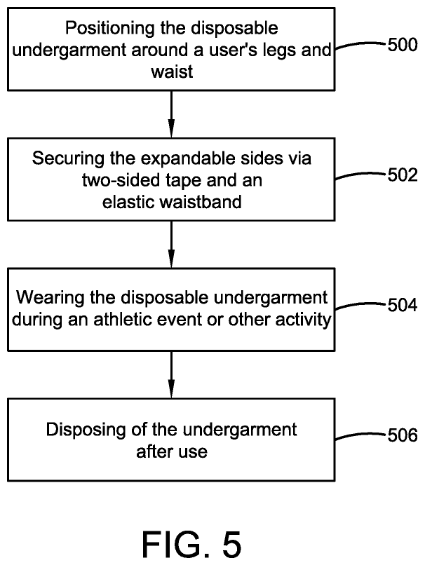

FIG. 5 illustrates a flowchart of the method of providing users with a disposable undergarment capable of being worn underneath clothing during athletic events. The method includes the steps of at 500, positioning the disposable undergarment around a user's legs and waist, wherein the disposable undergarment comprises a waist opening and a pair of leg openings. Further, the waist opening comprises an elastic waistband including expandable sides which are secured in place by two-sided tape. The expandable sides allow for customization of the undergarment, allowing a user to secure the waistband around their waist. The method also comprises at 502, securing the expandable sides via two-sided tape and an elastic waistband. A user adjusts the sides in position and then secures then via the tape, or other securing means. Further, the method comprises at 504, wearing the disposable undergarment during an athletic event or other activity. The method also comprises wearing the disposable undergarment for hospital use, military use, by handicap individuals who may struggle to make it to the restroom in a timely manner, and/or by a senior who desires a more fashionable disposable underwear. A user wears the disposable undergarment as they would a standard undergarment and is able to participate in various athletic events or other activities. Finally, at 506, disposing of the undergarment after use. A user can then throw-out the disposable undergarment after use and can choose a new disposable undergarment for the next activity if needed.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “disposable undergarment device”, “undergarment device”, and “device” are interchangeable and refer to the disposable undergarment device 100 of the present invention.

Notwithstanding the foregoing, the disposable undergarment device 100 of the present invention can be of any suitable size and configuration as is known in the art without affecting the overall concept of the invention, provided that it accomplishes the above-stated objectives. One of ordinary skill in the art will appreciate that the disposable undergarment device 100 as shown in FIGS. 1-5 are for illustrative purposes only, and that many other sizes and shapes of the disposable undergarment device 100 are well within the scope of the present disclosure. Although the dimensions of the disposable undergarment device 100 are important design parameters for user convenience, the disposable undergarment device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A disposable undergarment device for protecting against a bodily leak, the disposable undergarment device comprising:
an undergarment comprising a body portion, a waist opening and a pair of leg openings;
wherein an inside of the undergarment body comprises an absorbent padding to absorb the bodily leak;
wherein the undergarment body can be worn during an athletic event and other activities; and
further wherein the waist opening is positioned at a top of the undergarment, and the pair of leg openings are positioned at a bottom of the undergarment.

2. The disposable undergarment device of claim 1, wherein the undergarment is shaped as a male boxer brief.

3. The disposable undergarment device of claim 1, wherein the undergarment is shaped as a female undergarment.

4. The disposable undergarment device of claim 1, wherein the undergarment comprises a plurality of stretchable portions positioned around the waist opening and the pair of leg openings.

5. The disposable undergarment device of claim 1, wherein the undergarment comprises at least one piece that is thermally bonded at a seam, mechanically sewn, or bonded with an adhesive.

6. The disposable undergarment device of claim 5, wherein the body portion comprises a front panel and a back panel with an interconnecting crotch region all secured together.

7. The disposable undergarment device of claim 5 wherein the body portion comprises a single unitary structure generally have an hourglass shape, and further wherein a front panel and a back panel are integrally joined with an interconnecting crotch region.

8. The disposable undergarment device of claim 1, wherein the body portion further comprises an elastic waistband.

9. The disposable undergarment device of claim 8, wherein the elastic waistband comprises a pair of expandable sides.

10. The disposable undergarment device of claim 9, wherein the pair of expandable sides are secured together via a two-sided tape.

11. The disposable undergarment device of claim 1, wherein the undergarment comprises a layered structure having a soft hydrophilic rayon fabric layer disposed inside the undergarment and facing a user's skin.

12. The disposable undergarment device of claim 11 further comprising a separate cellulosic defense panel for moisture treatment within the undergarment.

13. The disposable undergarment device of claim 12, wherein the separate cellulosic defense panel is positioned toward a front area for a male and between a female's thighs.

14. The disposable undergarment device of claim 1, wherein the undergarment is formed from a plurality of natural fibers, a plurality of synthetic fibers, or a combination of a plurality of natural and synthetic fibers.

15. The disposable undergarment device of claim 14, wherein the undergarment further comprises a fabric having a hydrophilic site and a hydrophobic site to absorb moisture and repel water as needed.

16. A disposable undergarment device for protecting against a bodily leak, the disposable undergarment device comprising:
an undergarment comprising a body portion, a waist opening and a pair of leg openings; and
wherein the body portion comprises a front panel and a back panel with an interconnecting crotch region all secured together;
wherein the undergarment further comprises a layered structure with a soft hydrophilic rayon fabric layer disposed inside the undergarment and facing a user's skin, and a separate cellulosic defense panel for moisture treatment;
wherein the separate cellulosic defense panel is positioned toward a front area for a male and between a female's thighs; and
further wherein the waist opening comprises an elastic waistband with a pair of expandable sides.

17. The disposable undergarment device of claim 16, wherein the undergarment comprises a plurality of indicia.

18. The disposable undergarment device of claim 16, wherein the undergarment is formed from a plurality of natural fibers, a plurality of synthetic fibers, or a combination of a plurality of natural and synthetic fibers.

19. The disposable undergarment device of claim 18, wherein the undergarment further comprises a hydrophobic site to absorb moisture and repel water as needed.

20. A method of providing a user with a disposable undergarment capable of being worn underneath clothing during an athletic event or other activity, the method comprising the steps of:
positioning the disposable undergarment around a user's legs and waist;
securing a pair of expandable sides of the disposable undergarment with a two-sided tape and an elastic waistband;
wearing the disposable undergarment during the athletic event or other activity; and
disposing of the disposable undergarment after use.
